(19) United States
(12) Patent Application Publication
(10) Pub. No.: US 2011/0166529 A1
(43) Pub. Date: Jul. 7, 2011

(54) Medical appliance securing device

(76) Inventors: Matthew J. LeLievre, Estero, FL (US); Randel B. Holmes, Knoxville, TN (US)

(21) Appl. No.: 12/927,410

(22) Filed: Nov. 12, 2010

Related U.S. Application Data
(60) Provisional application No. 61/281,090, filed on Nov. 12, 2009.

(51) Int. Cl. A61M 25/02

(52) U.S. Cl. 604/180; 604/174

(57) ABSTRACT

A device for securing a medical appliance, such as an intravenous catheter hub. According to one embodiment, the securing device includes an anchor. The anchor includes a base layer and an adhesive layer, the adhesive layer being applied to the bottom surface of the base layer. A pair of foam blocks are adhered to the bottom surface of the adhesive layer of the anchor. The blocks are arranged to snugly receive the catheter hub and have a height substantially equal to that of the catheter hub. A transverse slit is provided in the anchor above the space between the blocks, the transverse slit being dimensioned to receive the nipple of the catheter hub. Reinforcing material may be mounted on the top surface of the anchor to minimize expansion of the transverse slit when the nipple is inserted through it. A pair of peelable release liners cover the bottom of the anchor and the blocks.

### CROSS-REFERENCE TO RELATED APPLICATIONS

The present application claims the benefit under 35 U.S.C. 119(e) of U.S. Provisional Patent Application Ser. No. 61/281,090, filed Nov. 12, 2009, the disclosure of which is incorporated herein by reference.

### BACKGROUND OF THE INVENTION

The present invention relates generally to medical appliance securing devices and more particularly to a novel medical appliance securing device.

Various medical devices, or portions thereof, are shaped as generally tubular members. Examples of such devices include, but are not limited to, catheters, catheter hubs, thermometers, intravenous needles, intravenous needle hubs, and various types of electrical wires. There is often a need to secure such devices to or near a patient's body so that the devices can appropriately function. For example, catheters, which are generally long, tubular, flexible conduits used to transport various types of fluids to and from the body of a patient, are often laterally and/or longitudinally fixed to the body of a patient to ensure proper placement and functioning of the catheter, as well as to offer some degree of comfort and mobility for the patient. In particular, some medical procedures involving the use of catheters require the application of a tensile force, known as traction, to the catheter, thus making it particularly necessary to firmly secure the catheter in its longitudinal direction.

To secure a catheter, a hub, or other medical appliance in the manner indicated above, practitioners have often used one or more strips of conventional, medical-grade, adhesive tape to secure a length of the medical appliance directly to the patient's skin. However, this method has its shortcomings as the adhesive tape tends to become loose over time. In addition, each time that one wishes to adjust the placement of the medical appliance or each time that one wishes to remove the medical appliance from the patient, the adhesive tape must be removed from the skin of the patient, thereby frequently causing irritation and discomfort to the patient.

Moreover, where the medical appliance is an intravenous catheter or the like, it is important that the catheter be secured on the patient in such a way as to minimize side-to-side rolling movement of the catheter, particularly when the patient moves or when a feeding set or other medical appliance is connected to the catheter, typically via a hub located at the proximal end of the catheter. If such rolling movement is not minimized, the catheter may cause inflammation of the patient's vein (i.e., phlebitis). Typically, securement is attempted by applying a strip of tape over the catheter hub and the patient's skin. However, as can be appreciated, because the catheter hub is tubular, the strip of tape typically contacts the skin at points spaced away from the catheter hub, thereby enabling the catheter hub some freedom to roll. This problem is often exacerbated by the fact that the catheter hub typically includes a protrusion or nipple facing upwardly. This nipple is intended to assist a health provider in keeping the hub stationary when an intravenous needle previously used to access the vein is removed from the hub, but it also has the unintended effect of providing a standoff that minimizes the surface area of the hub that may be contacted with the strip of tape. As can be appreciated, the less surface area contacted with the strip of tape, the more free the hub will be to roll.

Various types of medical appliance securing devices have been devised in an effort to provide the desired restraint and to overcome the disadvantages associated with the use of strips of adhesive tape. For example, in U.S. Pat. No. 4,165,748, inventor Johnson, which issued Aug. 28, 1979, and which is incorporated herein by reference, there is disclosed a simple, easily applied, catheter securing device. The catheter securing device includes two main parts having adhesive thereon for temporary attachment to the limb of a patient. In addition, the device includes a narrow bridge connecting the two main parts. The narrow bridge is also provided with adhesive and is foldable on itself to form a double member. Fasteners, such as snap fasteners or VELCRO® hook and loop fasteners, are positioned on the narrow bridge in such a way that the catheter tube may be releasably held by the narrow bridge in a desired orientation to a patient.

Another example of a medical appliance securing device is disclosed in U.S. Pat. No. 4,976,700, inventor Tollini, which issued Dec. 11, 1990, and which is incorporated herein by reference. In this patent, there is disclosed a securing device for securing to a patient's skin or to a support, a medical device such as tubing, a catheter, an intravenous needle, or the like, including an elongated tape having base portions and a central tab formed integrally therewith, pressure-sensitive tape on the base portions and on an exposed window of the tab, and hook and pile fastener portions on opposite sides of the exposed adhesive on the tab and on the base portion facing the exposed adhesive. A method of fabricating a securing device consisting of the steps of providing a strip of pressure-sensitive tape with release paper thereon, cutting out a window in a central portion of the strip of tape, removing release paper from the central portion of the pressure-sensitive tape, folding the central portion on itself to cause the facing exposed pressure-sensitive adhesive parts to adhere to each other and to provide a window of pressure-sensitive tape defined by the window which was cut out with the remainder of the strip forming a base, and securing hook and pile fastener material on opposite sides of the window of pressure-sensitive tape on the tab and on the portion of the securing device adjacent thereto which constitutes a base.

Still another example of a medical appliance securing device is disclosed in U.S. Pat. No. 5,147,322, inventors Bowen et al., which issued Sep. 15, 1992, and which is incorporated herein by reference. In this patent, there is disclosed a securing device for laterally and longitudinally securing generally tubular members having various diameters to any desired location on the surface of a patient's skin or other support. The device comprises an anchoring patch having one surface coated with adhesive for bonding the device to a patient's skin or some other support. A retaining tab is connected to the anchoring patch and contains an aperture such that the retaining tab may be wrapped around the circumference of the tubular member, inserted through the aperture, and firmly secured to the anchoring patch through the use of fastening means.

Still yet another example of a medical appliance securing device is disclosed in U.S. Pat. No. 5,304,146, inventors Johnson et al., which issued Apr. 19, 1994, and which is incorporated herein by reference. In this patent, there is disclosed a device for securing a generally tubular member of a medical appliance to a support surface. In one embodiment, the device comprises an anchoring patch, the anchoring patch including a first segment and a second segment and having a top surface and a bottom surface. The bottom surface is coated with an adhesive for attaching the anchoring patch to the support surface. A pair of flexible retaining tabs extend from and interconnect the inner edges of the first and second segment, the flexible retaining tabs being of sufficient length to helically wrap around the circumference of the generally tubular member and contact the top surface of the anchoring patch. First fasteners are secured to the free ends of the flexible retaining tabs and a pair of complementary fasteners are spaced outwardly relative to the tabs and are secured to the top surface of said anchoring patch. A generally tubular member may be retained in the device either by helically wrapping the flexible retaining tabs around the circumference of the generally tubular member and then coupling together the fasteners or by positioning the generally tubular member between the flexible tabs and the complementary fasteners and looping the retaining tabs over the generally tubular member and then coupling together the fasteners.

Still even yet another example of a medical appliance securing device is disclosed in U.S. Pat. No. 6,419,660, inventor Russo, which issued Jul. 16, 2002, and which is incorporated herein by reference. In this patent, there is disclosed a tube holder and a method for manufacturing the tube holder. The tube holder includes a base for attachment to a surface, for example, a patient's skin, and a tab for securing the tube to the base. According to one embodiment, the tube holder includes a first layer having first and second sides and first and second sections, and a second layer having first and second sides and first and second sections. The first sides of the first and second layers are attached to one another in the first sections of the first and second layers, the second sections of the first sides of the first and second layers are unattached to one another, and the first sections of the first and second layers form the tab and the second sections form the base. The tube holder also includes a third layer attached to the second side of the first layer for receiving a tube.

Still a further example of a medical appliance securing device is disclosed in U.S. Patent Application Publication No. US 2006/0041233 A1, inventor Bowen, which was published Feb. 23, 2006, and which is incorporated herein by reference. In this patent application, there is disclosed an apparatus for releasably securing an appliance on or adjacent a person. The apparatus includes a base and a flap or tongue attached to the base. The tongue may be wrapped around portions of an appliance and then attached to the base to releasably secure the appliance to the base. The base is formed from a first layer of material. The tongue is formed from a second layer of material disposed on the first layer. One or more of the layers is formed from medical grade adhesive tape or any other type of generally flexible material compatible with placement on a person's skin. Some embodiments may include multiple tongues to releasably secure one appliance or multiple appliances to a single base.

Still yet a further example of a medical appliance securing device is disclosed in U.S. Design Pat. No. 608,887, inventors Kyvik et al., which issued Jan. 26, 2010. In this patent, there is disclosed a catheter anchor pad with a release layer.

An example of a commercially available medical appliance securing device is the CATH-SECURE™ medical appliance securing device, which is available from the present assignee, M.C. Johnson Company, Inc. (Naples, Fla.). The CATH-SECURE™ medical appliance securing device, which corresponds generally to the device of U.S. Pat. No. 4,165,748, is formed by cutting a sheet of DURAPORE® surgical tape into two anchoring patches interconnected by a narrow bridge. (DURAPORE® surgical tape, which is commercially available from 3M Corporation (St. Paul, Minn.), is a tape consisting of a woven, polyester cloth backing having an acrylate pressure-sensitive adhesive applied to one surface thereof.) The narrow bridge is then folded onto and adhered to itself to form a double member retaining tab. VELCRO® hook and loop fasteners are then adhered to the double member retaining tab at locations such that a medical tube may be releasably held by the retaining tab in a desired orientation to a patient.

Another example of a commercially available medical appliance securing device is the CATH-SECURE Plus™ medical appliance securing device, which is available from the present assignee, M.C. Johnson Company, Inc. (Naples, Fla.). The CATH-SECURE Plus™ medical appliance securing device differs principally from the CATH-SECURE medical appliance securing device in that the CATH-SECURE Plus™ medical appliance securing device is water-resistant and has a butterfly-shaped anchor pad.

Still another example of a commercially available medical appliance securing device is the GRIP-LOK® universal securement device, which is available from Zefon International, Inc. of Ocala, Fla. The GRIP-LOK® universal securement device corresponds substantially to the device disclosed in the above-mentioned U.S. Design Pat. No. 608,887.

### SUMMARY OF THE INVENTION

It is an object of the present invention to provide a novel medical appliance securing device.

According to one aspect of the invention, there is provided a medical appliance securing device, said medical appliance securing device comprising (a) an anchor, said anchor being adapted to be secured to a surface; (b) a strip, said strip being positioned over said anchor and having a first end and a second end; (c) means for releasably securing said second end of said strip to said anchor; and (d) a cover, said cover being positioned over said anchor and said strip, with said first end of said strip fixedly secured between said cover and said anchor.

According to another aspect of the invention, there is provided a medical appliance securing device, said medical appliance securing device comprising (a) an anchor, said anchor comprising an adhesive bottom surface; (b) a pair of blocks adhered to said adhesive bottom surface of said anchor, said pair of blocks being arranged to snugly receive a medical appliance therebetween; (c) at least one release liner adhered to said adhesive bottom surface of said anchor.

According to still another aspect of the invention, there is provided a medical appliance securing device, said medical appliance securing device comprising (a) an anchor, said anchor comprising an adhesive bottom surface; (b) a one-piece member adhered to said adhesive bottom surface of said anchor, said one-piece member being shaped to comprise a pair of blocks interconnected by a wall, said one-piece member being dimensioned to snugly receive a medical appliance between said pair of blocks; (c) at least one release liner adhered to said adhesive bottom surface of said anchor.

According to still yet another aspect of the invention, there is provided a medical appliance securing device, said medical appliance securing device comprising (a) an anchor, said anchor comprising an adhesive bottom surface; (b) a double-sided adhesive patch, said double-sided adhesive patch being adhered to a top surface of said anchor; (c) a pair of blocks adhered to a top surface of said double-sided adhesive patch, said pair of blocks being arranged to snugly receive a medical appliance therebetween; and (d) at least one release liner adhered to said adhesive bottom surface of said anchor.

The present invention is also directed at methods of using the above-described medical appliance securing devices.

For purposes of the present specification and claims, various relational terms like “top,” “bottom,” “proximal,” “distal,” “upper,” “lower,” “front,” and “rear” are used to describe the present invention when said invention is positioned in or viewed from a given orientation. It is to be understood that, by altering the orientation of the invention, certain relational terms may need to be adjusted accordingly.

Additional objects, as well as features and advantages, of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. In the drawings wherein like reference numerals represent like parts:

FIG. 1 is a top view of a first embodiment of a medical appliance securing device constructed according to the teachings of the present invention;

FIG. 2 is a side view of the medical appliance securing device shown in FIG. 1 (with certain components not being drawn to scale for illustrative purposes);

FIG. 3 is a partially exploded perspective view of the medical appliance securing device shown in FIG. 1;

FIGS. 4(a) through 4(c) are side views (with certain components not being drawn to scale for illustrative purposes), showing how the medical appliance securing device of FIG. 1 may be used;

FIGS. 5(a), 5(b), and 5(c) are top, bottom, and partially exploded side views, respectively, of a second embodiment of a medical appliance securing device constructed according to the teachings of the present invention (with certain components not being drawn to scale for illustrative purposes);

FIG. 6 is a bottom view of the medical appliance securing device of FIGS. 5(a) through 5(c) with the peelable release liners not being shown;

FIGS. 7(a) and 7(b) are top and side views, respectively, showing how the medical appliance securing device of FIGS. 5(a) through 5(c) may be used (with certain components not being drawn to scale or shown for illustrative purposes and with the stop blocks being shown in phantom in FIG. 7(a));

FIG. 8 is a partially exploded side view of a third embodiment of a medical appliance securing device constructed according to the teachings of the present invention (with certain components not being drawn to scale for illustrative purposes);

FIG. 9 is a partially exploded side view of fourth embodiment of a medical appliance securing device constructed according to the teachings of the present invention (with certain components not being drawn to scale for illustrative purposes);

FIG. 10 is a top view of the block shown in FIG. 9;

FIGS. 11 and 12 are top and partially exploded side views, respectively, of a fifth embodiment of a medical appliance securing device constructed according to the teachings of the present invention (with certain components not being drawn to scale for illustrative purposes); and

FIG. 13 is a top view showing how the medical appliance securing device of FIGS. 11 and 12 may be used.

### DETAILED DESCRIPTION OF PREFERRED EMBODIMENTS

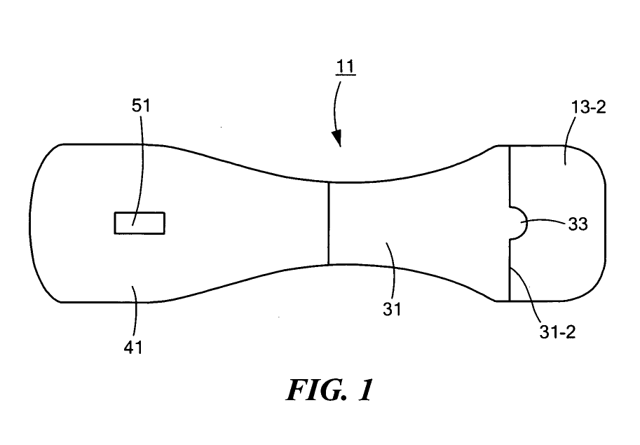

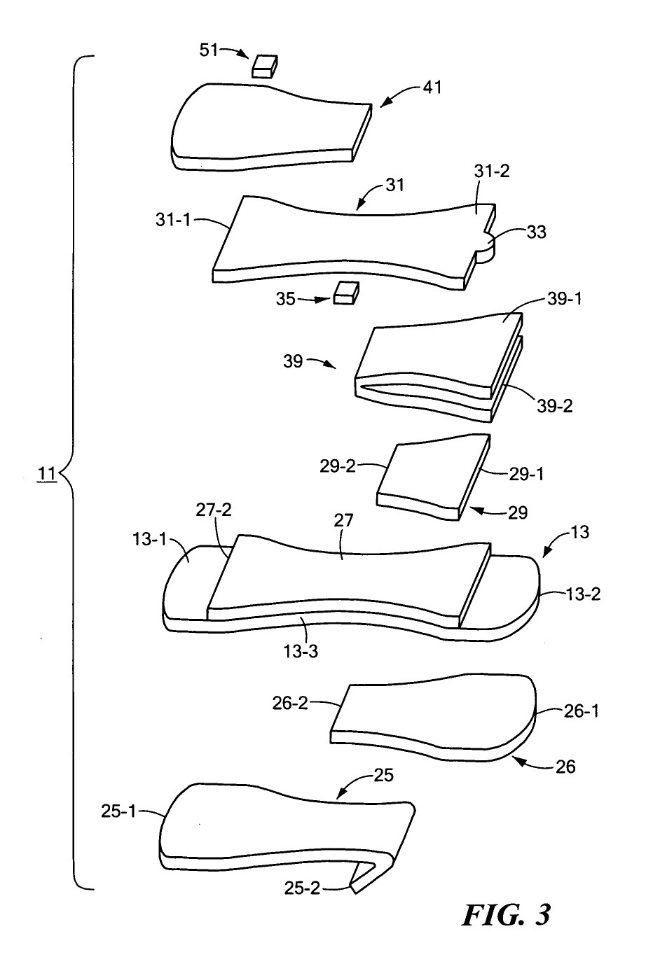

Referring now to FIGS. 1 through 3, there are shown various view of a first embodiment of a medical appliance securing device constructed according to the teachings of the present invention, said medical appliance securing device being represented generally by reference numeral 11. In the present embodiment, device 11 is particularly well-suited for securing an intravenous tube; however, device 11 is not intended to be limited to this use and may also be used to secure other types of tubular members, such as an intravenous catheter hub.

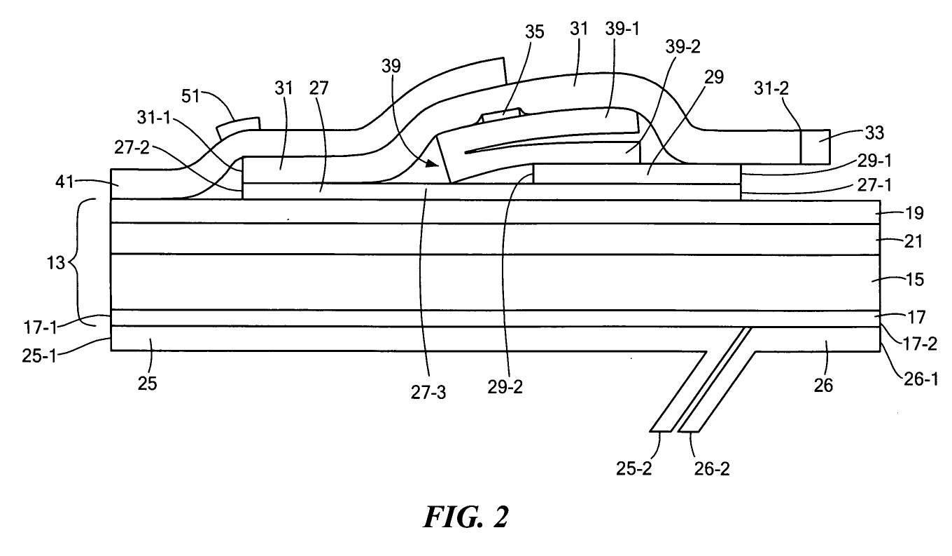

Device 11 may comprise an anchor 13. When viewed from above, anchor 13 may have a generally hourglass profile that may include a pair of generally-rectangular end patches 13-1 and 13-2 interconnected by a biconcave bridge 13-3. Anchor 13 may be a multi-layered structure (see FIG. 2) that may include a base layer 15, an adhesive layer 17, and a water barrier layer 19. Base layer 15 may be a layer of breathable fabric and may include, for example, a layer of SONTARA® non-woven, spun-laced, hydro-entangled, polyester fabric having a basis weight of about 1.2 oz/yd² (E.I. du Pont de Nemours and Company, Wilmington, Del.). Adhesive layer 17, which may be applied directly to the bottom surface of base layer 15, may comprise a pressure-sensitive adhesive and may be, for example, an acrylic pressure-sensitive adhesive having a thickness of about 0.001 inch. Barrier layer 19, which may be positioned over the top surface of base material 15 and may be adhered thereto using a tie layer 21, may be a breathable material that is also water-resistant, i.e., possesses a high moisture vapor transmission rate (MVTR). For example, barrier layer 19 may be a layer of monolithic polyurethane having a thickness of about 0.001 inch. The aforementioned monolithic polyurethane may be, for example, a non-foamed, caromatiac polyether type having a Shore hardness A of 83; a specific gravity of 1.13; a 100% module-less of 1100; a 300% module-less of 2700; an ultimate tensile of 9000 (elasticity); and a tear resistance of 625.

As noted above, tie layer 21 serves to adhere barrier layer 19 to base layer 15. Accordingly, where barrier layer 19 is a layer of monolithic polyurethane of the type described above and base layer 15 includes a layer of SONTARA® non-woven, spun-laced polyester fabric, tie layer 21 may be, for example, an acrylic adhesive having a thickness of about 0.0005 inch.

An example of a multi-layered material suitable for use in forming anchor 13 is commercially available from DermaMed Coatings Company, LLC (Tallmadge, Ohio) as DM-6001 tape. DM-6001 tape includes a base layer of SONTARA® non-woven, spun-laced, hydro-entangled, polyester fabric having a basis weight of about 1.2 oz/yd², an acrylic pressure-sensitive adhesive layer having a thickness of about 0.001 inch applied directly to the bottom surface of the base layer, an acrylic adhesive tie layer having a thickness of about 0.0005 inch applied directly to the top surface of the base layer, and a monolithic polyurethane barrier layer having a thickness of about 0.001 inch applied directly to the top surface of the tie layer. Using ASTM E96 (an industry standard method for assessing moisture vapor transmission rate), DM-6001 tape has an upright MVTR of 474 grams/m² over a 24-hour period and an inverted MVTR of 576 grams/m² over a 24-hour period.

Peelable release liners 25 and 26 may be removably adhered to the bottom of anchor 13. Liner 25 may cover a majority of the exposed bottom surface of adhesive layer 17, with a first end 25-1 of liner 25 lying flush with a first end 17-1 of adhesive layer 17 and with a second end 25-2 of liner 25 being folded away from adhesive layer 17 to facilitate its being peeled away by a user. Liner 26 may cover the remaining exposed bottom surface of adhesive layer 17, with a first end 26-1 of liner 26 lying flush with a second end 17-2 of adhesive layer 17 and with a second end 26-2 of liner 26 extending away from adhesive layer 17 to facilitate its being peeled away by a user. As will be discussed further below, when one wishes to adhere device 11 to a surface, liners 25 and 26 are peeled away from anchor 13 to expose adhesive layer 17.

Device 11 may additionally comprise an adhesive layer 27. Adhesive layer 27 may be applied directly to the top surface of water barrier layer 19 and may extend over the entirety of bridge 13-3, as well as nearby adjacent portions of patches 13-1 and 13-2.

Device 11 may additionally comprise a VELCRO® hook fastening patch 29. Patch 29 may be applied directly to the top surface of adhesive layer 27, with a first end 29-1 of patch 29 lying flush with end 27-1 of adhesive layer 27 and with a second end 29-2 of patch 29 terminating a short distance prior to the midpoint of adhesive layer 27.

Device 11 may additionally comprises a VELCRO® loop fastening strip 31, strip 31 being made of a material capable of detachably mating with patch 29. (As can be appreciated, the use of VELCRO® fastening elements for patch 29 and strip 31, while preferred, is not essential and other types of fastening mechanisms may also be used.) As viewed from above, strip 31 may be shaped similarly to adhesive layer 27. A little less than half of strip 31 may be secured directly to adhesive layer 27, with a first end 31-1 of the secured portion of strip 31 lying flush with and secured to end 27-2 of adhesive layer 27. A second end 31-2 of strip 31 is not secured to adhesive layer 27, but rather, extends slightly beyond patch 29 to facilitate the grasping of end 31-2 of strip 31 when one wishes to detach strip 31 from patch 29. To further facilitate the grasping of end 31-2 of strip 31, a tab 33 or other suitable structure (e.g., one or more nipples) may extend from end 31-2 in a direction away from end 31-1.

As can be appreciated, at the approximate midsection of adhesive layer 27, i.e., between patch 29 and the secured portion of strip 31, adhesive layer 27 is left exposed to define an adhesive patch 27-3. As will be apparent from the discussion below, the intravenous tube or other tubular member to be secured is preferably positioned directly on top of patch 27-3. To facilitate the identification of patch 27-3 to a user, patch 27-3 may be marked with identifying indicia. This may be accomplished by means of ink or other markings positioned below patch 27-3, provided that patch 27-3 is transparent. Alternatively, ink or other markings may be positioned within or above patch 27-3, or patch 27-3 may otherwise be textured or colored in some fashion to heighten its visibility.

Device 11 may additionally comprise an adhesive strip 35. Adhesive strip 35, which may be about ⅛ inch in width, may be applied directly to the bottom surface of fastening strip 31 and may extend across the width of fastening strip 31 at a location approximating the midpoint of the length of fastening strip 31. In this manner, strip 35 may be aligned with patch 27-3 of adhesive layer 27. To facilitate the identification of strip 35 to a user, strip 35 may be marked with identifying indicia. This may be accomplished by means of ink or other markings positioned above strip 35, provided that strip 35 is transparent. Alternatively, ink or other markings may be positioned within or below strip 35, or strip 35 may otherwise be textured or colored in some fashion to heighten its visibility.

Device 11 may additionally comprise a peelable release liner 39. Liner 39, which may be shaped similarly to bridge 13-3, may be folded to yield a top portion 39-1 and a bottom portion 39-2. Top portion 39-1 may be positioned so as to cover strip 35 on fastening strip 31, and bottom portion 39-2 may be positioned so as to cover patch 27-3. As will be discussed further below, when one wishes to adhere device 11 to a tubular member, liner 39 is peeled away to expose adhesive strip 39 and adhesive patch 27-3.

Device 11 may additionally comprise a cover layer 41. Layer 41, which may be, for example, a polyurethane coating, may be positioned directly over and secured to the fixed end of strip 31, as well as to the adjacent exposed portion of patch 13-1. In fact, layer 41 preferably covers approximately the fixed half of strip 31, as well as the adjacent exposed portion of patch 13-1. The purpose of layer 41 is to prevent the fixed end of strip 31 from inadvertently becoming separated from anchor 13 when the free end of strip 31 is pulled, with great force, away from patch 29.

Device 11 may additionally comprise a fastening element 51 fixed to the top surface of layer 41 for releasably engaging end 31-2 of strip 31. In this manner, element 51 may be used to temporarily maintain strip 31 in a folded-back state so as to prevent adhesive strip 35 from adhering to adhesive patch 27-3 after a user has removed liner 39 and before the user has positioned a tubular member between strip 35 and patch 27-3. Fastening element 51 may be, for example, a patch of repositionable adhesive. (Alternatively, fastening element 51 may be a VELCRO® fastener, with a complementary VELCRO® fastener appropriately positioned on strip 31. Other fastening mechanisms will be apparent to those of ordinary skill in the art.)

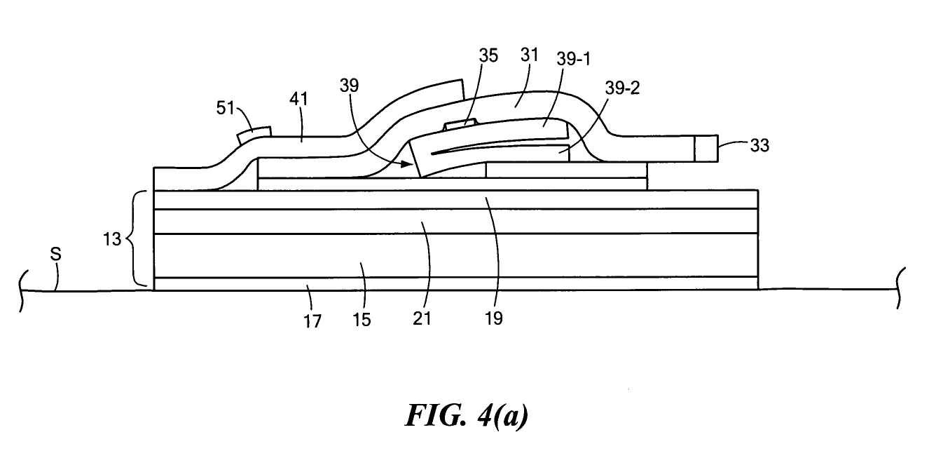

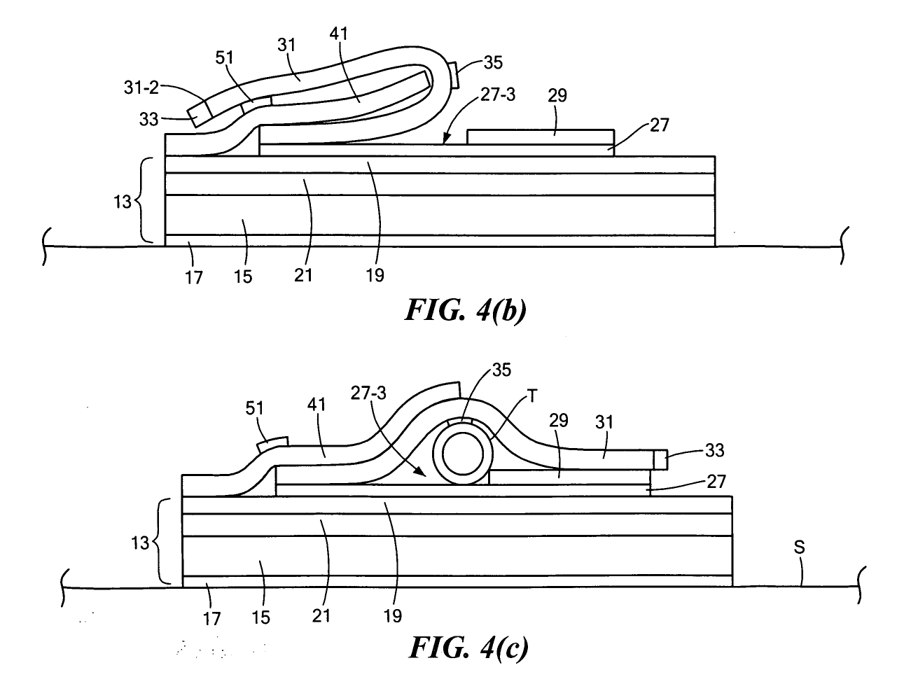

Referring now to FIGS. 4(a) through 4(c), device 11 may be used as follows: First, liners 25 and 26 are peeled away from anchor 13, and adhesive layer 17 is secured to a patient's skin S or to another suitable surface (see FIG. 4(a)). Next, end 31-2 of strip 31 is detached from patch 29 and is folded back and secured to cover 41 using fastening element 51. Liner 39 is then removed both from strip 35 and from adhesive patch 27-3 (see FIG. 4(b)). As can be seen, because of the folding back of strip 31, strip 35 is prevented from inadvertently coming into contact with patch 27-3, which contact would be undesirable. Next, an intravenous tube T or other tubular member is positioned on top of patch 27-3, strip 31 is detached from fastening element 51 and is unfolded so that adhesive strip 35 is brought into contact against the top of tube T, and end 31-2 of strip 31 is then secured to patch 29 to securely hold tube T in place (see FIG. 4(c)). When retention of tube T is no longer desired, end 31-2 of strip 31 may be removed from patch 29, and tube T may be pulled away from patch 27-3 and strip 35.

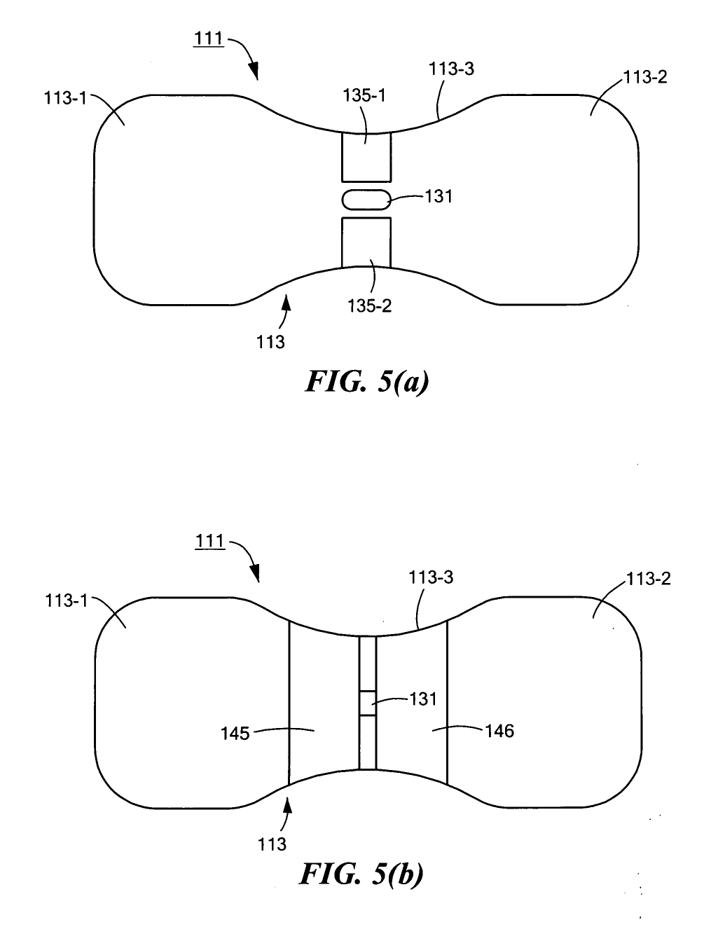

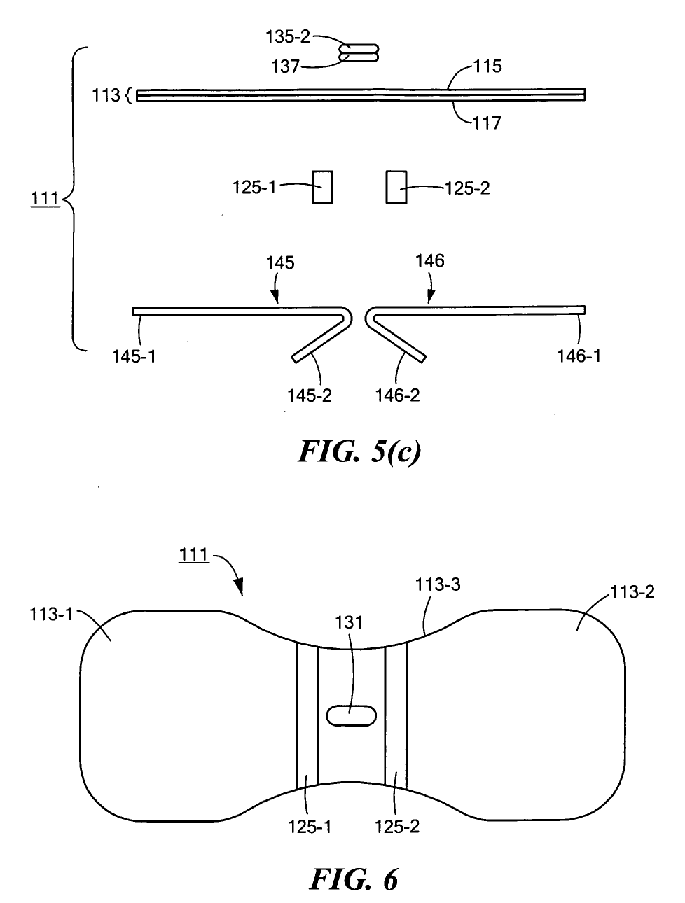

Referring now to FIGS. 5(a), 5(b), 5(c) and 6, there are shown various views of a second embodiment of a medical appliance securing device constructed according to the teachings of the present invention, the medical appliance securing device being represented generally by reference numeral 111. In the present embodiment, device 111 is particularly well-suited for securing a conventional intravenous catheter hub; however, device 111 is not intended to be limited to this use and may also be used to secure other types of tubular members.

Device 111 may comprise an anchor 113. When viewed from above, anchor 113 may have a generally hourglass profile that may include a pair of generally-rectangular end patches 113-1 and 113-2 interconnected by a biconcave bridge 113-3. Anchor 113 may comprise a strip of one-sided tape (see FIG. 5(c)), and, although not shown as such, anchor 113 may be transparent. Anchor 113 may include a base layer 115 and an adhesive layer 117. Base layer 115 may comprise a strip of a plastic film of the type conventionally used as the substrate in adhesive tapes. Adhesive layer 117, which may be applied directly to the bottom surface of base layer 115, may comprise a medical-grade, pressure-sensitive adhesive.

Device 111 may additionally comprise a pair of blocks 125-1 and 125-2 adhered to the bottom surface of adhesive layer 117 and arranged parallel to one another, blocks 125-1 and 125-2 being disposed under biconcave bridge 113-3 and extending generally transversely relative to the length of anchor 113. Blocks 125-1 and 125-2 may be appropriately spaced apart from one another to snugly receive therebetween a conventional intravenous catheter hub, and blocks 125-1 and 125-2 may have a height that approximates that of a conventional intravenous catheter hub. In this manner, blocks 125-1 and 125-2 may serve as stops between anchor 113 and the patient to minimize the freedom of a conventional intravenous hub to roll on a patient. Blocks 125-1 and 125-2 may be made of a foam, such as a closed cell foam or an open cell foam, a wicking foam or a non-wicking foam, a compressible foam or a non-compressible foam. In addition, blocks 125-1 and 125-2 may be impregnated with silver alginate or another suitable antimicrobial agent to reduce the risk of infection at the insertion site.

A transverse slit 131 may be provided in anchor 113, slit 131 extending longitudinally between blocks 125-1 and 125-2. Slit 131 may be appropriately dimensioned (e.g., 0.0625 inch wide) to receive the nipple of a conventional intravenous catheter hub. In this manner, the surface area of anchor 113 in contact with the hub may be maximized.

Device 111 may also comprise a pair of patches of reinforcing material 135-1 and 135-2 adhered with a medical-grade adhesive 137 to the top surface of anchor 113 in the area between blocks 125-1 and 125-2 and on opposite sides of slit 131. Patches 135-1 and 135-2, which may be made of a suitably rigid material, such as a 2-3 mil polyester or polyethylene, are intended to reinforce anchor 113 so as to keep slit 131 from opening wider as a result of tearing caused by the nipple of the hub.

Device 111 may further comprise a pair of peelable release liners 145 and 146 removably adhered to the bottom of anchor 113, except in the vicinity of blocks 125-1 and 125-2. Liner 145 may cover approximately half of the footprint of device 111, with a first end 145-1 of liner 145 lying flush with the end of patch 113-1 and with a second end 145-2 of liner 145 being folded away to facilitate its being peeled away by a user. Liner 146 may cover the remaining half of the footprint of device 111, with a first end 146-1 of liner 146 lying flush with the end of patch 113-2 and with a second end 146-2 of liner 146 being folded away to facilitate its being peeled away by a user.

If desired, adhesive may be applied to the bottom surfaces of blocks 125-1 and 125-2 to keep blocks 125-1 and 125-2 adhered to release liners 145 and 146, respectively, prior to use, for example, during storage or shipping. Such adhesive may also be desirable to keep blocks 125-1 and 125-2 adhered to a patient during use.

As can be appreciated, although not shown, anchor 113 could comprise, instead of base layer 115 and adhesive layer 117, the multi-layer structure of anchor 13 of device 11, namely, the combination of base layer 15, adhesive layer 17, water barrier layer 19 and tie layer 21, such as DM-6001 tape (DermaMed Coatings Company, LLC, Tallmadge, Ohio).

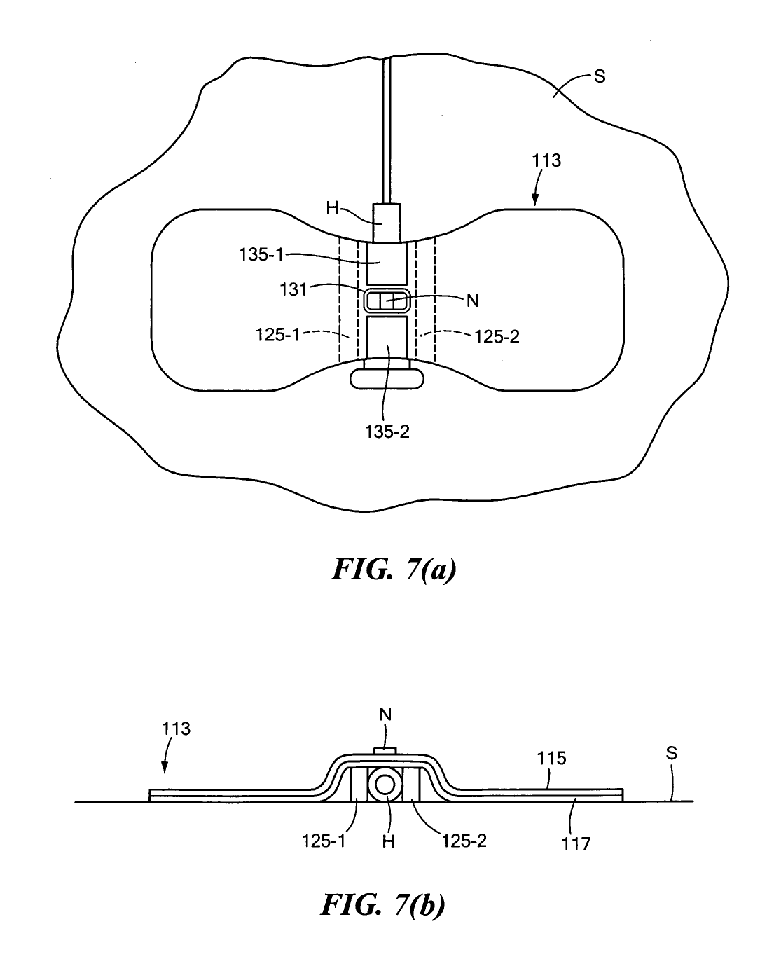

To use device 111, liners 145 and 146 may be peeled away from anchor 113 and away from blocks 125-1 and 125-2. Then, as seen in FIGS. 7(a) and 7(b), device 111 may then be positioned so that the hub H is seated between blocks 125-1 and 125-2, with nipple N extending through slit 131, and patches 113-1 and 113-2 may be adhered to the patient's skin S. In this manner, device 111 may be used to minimize rolling movement of hub H. When such restraint is no longer desired, device 111 may be pulled away from hub H and from the patient.

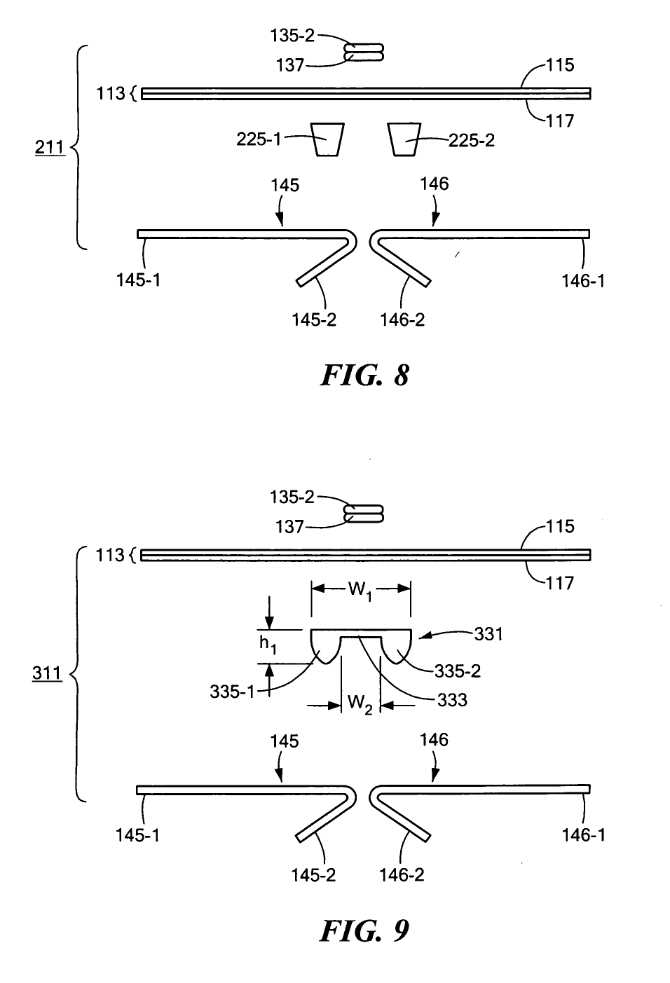

Referring now to FIG. 8, there is shown a partially exploded side view of a third embodiment of a medical appliance securing device constructed according to the teachings of the present invention, the medical appliance securing device being represented generally by reference numeral 211. In the present embodiment, device 211 is particularly well-suited for securing a conventional intravenous catheter hub; however, device 211 is not intended to be limited to this use and may also be used to secure other types of tubular members.

Device 211 may be similar in most respects to device 111, the principal difference between the two devices being that, whereas device 111 may include blocks 125-1 and 125-2 having a generally rectangular end profile, device 211 may include blocks 225-1 and 225-2 having a generally wedge-shaped end profile. In the present embodiment, this wedge-shape is trapezoidal, but it is not limited thereto.

Device 211 may be used in substantially the same manner as described above for device 111.

Referring now to FIG. 9, there is shown a partially exploded side view of a fourth embodiment of a medical appliance securing device constructed according to the teachings of the present invention, the medical appliance securing device being represented generally by reference numeral 311. In the present embodiment, device 311 is particularly well-suited for securing a conventional intravenous catheter hub; however, device 311 is not intended to be limited to this use and may also be used to secure other types of tubular members.

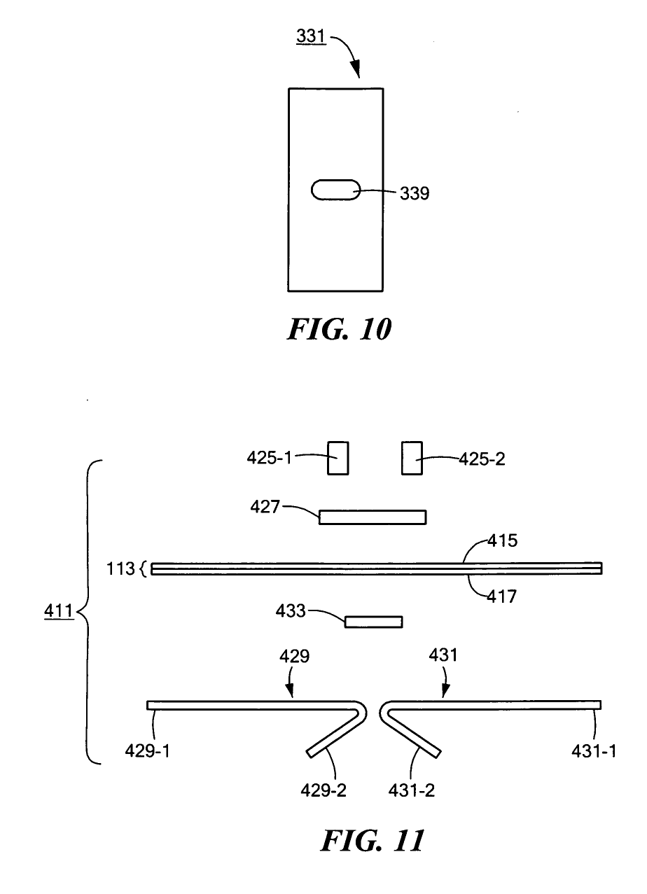

Device 311 may be similar in most respects to device 111, the principal difference between the two devices being that, whereas device 111 may include a pair of spaced apart blocks 125-1 and 125-2, device 311 may include a one-piece block 331. Block 331 may be made, for example, by extrusion molding and may be shaped to include a top wall 333 under which a hub may be positioned and a pair of side walls 335-1 and 335-2 for limiting rolling movement of the hub. Block 331 may be dimensioned, for example, to have an outside width w₁ of approximately 0.375 inch, an inside width w₂ of approximately 0.261 inch, and a height h₁ of approximately 0.135 inch. Block 331 may be shaped to comprise a slit 339 (see FIG. 10) that is aligned with slit 131 and through which the nipple of a hub may be inserted.

Device 311 may be used in substantially the same manner as described above for device 111.

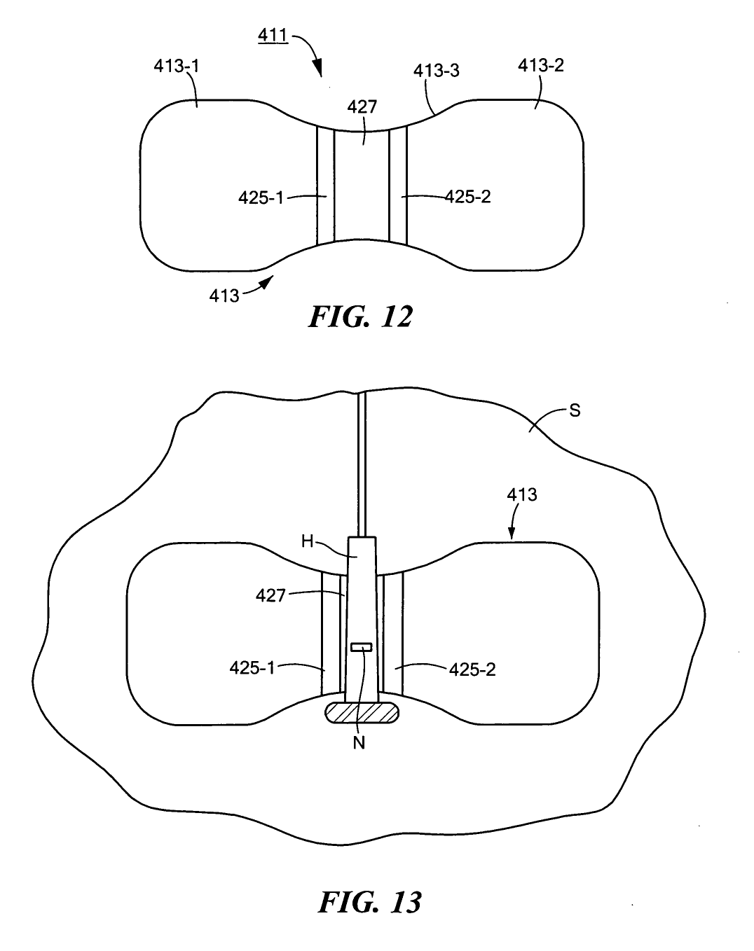

Referring now to FIGS. 11 and 12, there are shown top and partially exploded side views, respectively, of a fifth embodiment of a medical appliance securing device constructed according to the teachings of the present invention, the medical appliance securing device being represented generally by reference numeral 411. In the present embodiment, device 411 is particularly well-suited for securing a conventional intravenous catheter hub; however, device 411 is not intended to be limited to this use and may also be used to secure other types of tubular members.

Device 411 may comprise an anchor 413. When viewed from above, anchor 413 may have a generally hourglass profile that may include a pair of generally-rectangular end patches 413-1 and 413-2 interconnected by a biconcave bridge 413-3. Anchor 413 may comprise a base layer 415, which may be a layer of breathable fabric similar to that of base layer 15 of device 11. Anchor 413 may also comprise an adhesive layer 417, which may be the same as adhesive layer 17 of device 11 and which may be applied directly to the bottom surface of base layer 415.

Device 411 may additionally comprise a pair of blocks 425-1 and 425-2 adhered to the top surface of base layer 415 using a strip of double-sided tape 427. Blocks 425-1 and 425-2 may be constructed and dimensioned similarly to blocks 125-1 and 125-2 and may be arranged similarly to blocks 125-1 and 125-2, except that blocks 425-1 and 425-2 may be positioned above anchor 413 whereas blocks 125-1 and 125-2 may be positioned below anchor 113.

Device 411 may further comprise a pair of peelable release liners 429 and 431 removably adhered to the bottom of anchor 413, except in the central region of anchor 413 (for reasons to be explained below). Liner 429 may cover approximately half of the footprint of device 411, with a first end 429-1 of liner 429 lying flush with the end of patch 413-1 and with a second end 429-2 of liner 429 being folded away to facilitate its being peeled away by a user. Liner 431 may cover the remaining half of the footprint of device 411, with a first end 431-1 of liner 431 lying flush with the end of patch 413-2 and with a second end 431-2 of liner 431 being folded away to facilitate its being peeled away by a user.

Device 411 may additionally comprise a foam patch 433 adhered to the bottom surface of anchor 413, foam patch 433 having a similar footprint as tape 427. Foam patch 433 may serve to provide some cushioning to the patient for a medical appliance seated over anchor 413. In addition, because foam patch 433 covers adhesive layer 417 in the central region of anchor 413, the central region of anchor 413 is not adhesive along its bottom surface. As a result, a user may peel liners 429 and 431 just enough to expose patch 433 and then slide device 411 under the medical appliance to be secured, without running the risk of having the device adhere to the patient before the device is properly in position. Once device 411 is properly positioned, liners 429 and 431 may be removed entirely.

As noted above, to use device 411, liners 429 and 431 may be peeled away just enough to expose patch 433. Next, device 411 may be slid under the medical appliance, and the medical appliance may be pushed down between blocks 425-1 and 425-2 and adhered to the exposed portion of tape 427 (see FIG. 13). When securement is no longer desired, the medical appliance may be pulled away from device 411, and device 411 may be removed from the patient.

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A medical appliance securing device comprising:
(a) an anchor, said anchor being adapted to be secured to a surface;
(b) a strip, said strip being positioned over said anchor and having a first end and a second end;
(c) means for releasably securing said second end of said strip to said anchor; and
(d) a cover, said cover being positioned over said anchor and said strip, with said first end of said strip fixedly secured between said cover and said anchor.

2. The medical appliance securing device as claimed in claim 1 wherein said strip comprises a first fastening element and wherein said means for releasably securing said second end of said strip to said anchor comprises a second fastening element coupled to said anchor, said first and second fastening elements being complementary hook and loop fasteners.

3. The medical appliance securing device as claimed in claim 2 further comprising means for releasably securing said second end of said strip to a top surface of said cover.

4. The medical appliance securing device as claimed in claim 3 wherein said means for releasably securing said second end of said strip to said top surface of said cover comprises a third fastening element, said first and third fastening elements being complementary hook and loop fasteners.

5. The medical appliance securing device as claimed in claim 2 further comprising an adhesive patch disposed over said anchor between said first end of said strip and said second fastening element.

6. The medical appliance securing device as claimed in claim 5 wherein said adhesive patch is identified with identifying indicia.

7. The medical appliance securing device as claimed in claim 5 further comprising an adhesive strip applied to a bottom surface of said strip, said adhesive strip being aligned with said adhesive patch.

8. The medical appliance securing device as claimed in claim 7 wherein said adhesive strip is identified with identifying indicia.

9. The medical appliance securing device as claimed in claim 1 wherein said anchor comprises a fabric base layer, an adhesive layer, said adhesive layer positioned below said fabric base layer, and a water barrier layer, said water barrier layer being positioned above said fabric base layer.

10. A medical appliance securing device comprising:
(a) an anchor, said anchor comprising an adhesive bottom surface;
(b) a pair of blocks adhered to said adhesive bottom surface of said anchor, said pair of blocks being arranged to snugly receive a medical appliance therebetween;
(c) at least one release liner adhered to said adhesive bottom surface of said anchor.

11. The medical appliance securing device as claimed in claim 10 wherein said anchor comprises a transverse slit, said transverse slit being positioned above a space between said pair of blocks.

12. The medical appliance securing device as claimed in claim 11 further comprising reinforcing material positioned over said anchor adjacent to said transverse slit to minimize expansion of said transverse slit.

13. The medical appliance securing device as claimed in claim 10 wherein said pair of blocks are rectangular in end profile.

14. The medical appliance securing device as claimed in claim 10 wherein said pair of blocks are wedge-shaped in end profile.

15. The medical appliance securing device as claimed in claim 10 wherein said pair of blocks are foam blocks.

16. A medical appliance securing device comprising:
(a) an anchor, said anchor comprising an adhesive bottom surface;
(b) a one-piece member adhered to said adhesive bottom surface of said anchor, said one-piece member being shaped to comprise a pair of blocks interconnected by a wall, said one-piece member being dimensioned to snugly receive a medical appliance between said pair of blocks;
(c) at least one release liner adhered to said adhesive bottom surface of said anchor.

17. The medical appliance securing device as claimed in claim 16 wherein said anchor comprises a transverse slit, said transverse slit being positioned above a space between said pair of blocks.

18. The medical appliance securing device as claimed in claim 17 further comprising reinforcing material positioned over said anchor adjacent to said transverse slit to minimize expansion of said transverse slit.

19. The medical appliance securing device as claimed in claim 16 wherein said one-piece member is made by extrusion molding.

20. A medical appliance securing device comprising:
(a) an anchor, said anchor comprising an adhesive bottom surface;
(b) a double-sided adhesive patch, said double-sided adhesive patch being adhered to a top surface of said anchor;
(c) a pair of blocks adhered to a top surface of said double-sided adhesive patch, said pair of blocks being arranged to snugly receive a medical appliance therebetween; and
(d) at least one release liner adhered to said adhesive bottom surface of said anchor.

21. The medical appliance securing device as claimed in claim 20 further comprising a foam pad, said foam pad being adhered to the adhesive bottom surface of the anchor.

22. The medical appliance securing device as claimed in claim 21 wherein said anchor is generally hourglass-shaped, wherein said double-sided adhesive patch is centrally located on said anchor, and wherein said foam pad is centrally located on said anchor.

23. The medical appliance securing device as claimed in claim 22 wherein said at least one release liner comprises a pair of symmetrical release liners, each of said symmetrical release liners being peelable from the middle of said anchor.
